(19) United States
(12) Patent Application Publication
(10) Pub. No.: US 2008/0160533 A1
(43) Pub. Date: Jul. 3, 2008

(54) Assay for prediction of response to Met antagonists

(75) Inventors: Mark A. Hayden, Ingleside, IL (US); Jeffrey B. Huff, Park Ridge, IL (US); William E. Murray, Oak Park, IL (US)

Correspondence Address: VYSIS, INC;PATENT DEPARTMENT, 1300 E TOUHY AVENUE, DES PLAINES, IL, 60018, US

(73) Assignee: Abbott Laboratories, Abbott Park, IL (US)

(21) Appl. No.: 11/998,321

(22) Filed: Nov. 29, 2007

Related U.S. Application Data
(60) Provisional application No. 60/872,314, filed on Nov. 30, 2006.

(51) Int. Cl. C12Q 1/68

(52) U.S. Cl. 435/6

(57) ABSTRACT

A method for classifying cancer patients as likely to have lower response to MET receptor antagonist therapy comprises assessment of the presence or absence of a single nucleotide polymorphism in the MET promoter in a patient tissue sample. The invention provides more effective identification of patients to receive MET receptor antagonist therapy.

### FIELD OF THE INVENTION

This invention relates to diagnostic assays useful with MET receptor antagonist cancer therapy, and in particular relates to measurement of a single nucleotide polymorphism that allows identification of patients likely to exhibit lower response to MET receptor antagonist therapy.

### BACKGROUND OF THE INVENTION

P. Ma, et al., “Functional Expression and Mutations of c-Met and Its Therapeutic Inhibition with SU11274 and Small Interfering RNA in Non-Small Cell Lung Cancer”, Cancer Research 65, 1479-1488, Feb. 15, 2005, which is incorporated herein by reference, provide an excellent overview of the MET gene (citations within the following excerpt are omitted): “The c-Met gene is located on chromosome 7, band 7q31, and spans >120 kb long, consisting of 21 exons separated by 20 introns. In wild-type cells, the primary transcript produces a 150-kDa polypeptide, which gets partially glycosylated, and produces a 170-kDa precursor protein. This is further glycosylated and then cleaved to produce a 50-kDa α-chain and a 140-kDa β-chain, which are then linked by disulfide bonds. The ligand for c-Met has been identified as hepatocyte growth factor (HGF), also known as scatter factor. Signaling through the c-Met/HGF pathway has been shown to trigger a variety of cellular responses that may vary based on the cellular context. In vivo, c-Met/HGF signaling plays key role in growth, motility, invasion, metastasis, angiogenesis, wound healing, and tissue regeneration. Higher levels of HGF have also been associated with more aggressive biology and a worse prognosis in NSCLC [non-small cell lung cancer] and small cell lung cancer (SCLC). c-Met is normally expressed by epithelial cells and has been found to be overexpressed and amplified in a variety of human tumor tissues.” Id. at p. 1479.

Ma et al., is one example of MET receptor antagonist therapy, and also reports on the analysis of the full length of the MET gene for mutations potentially impacting MET inhibitor therapy. Ma et al., do not disclose nor suggest the identification of any mutations in the MET promoter, nor do they disclose nor suggest any single nucleotide polymorphisms in the MET promoter as impacting effectiveness of MET receptor antagonist therapy.

Other examples of MET inhibitor therapy for cancer are also described in the review C. Birchmeier et al., “MET, METASTASIS, MOTILITY AND MORE”, Nature Reviews, Molecular Cell Biology, 4 (December 2003): 915-925, which is incorporated herein by reference. These inhibitors include the antibiotic geldanmycin, small molecule inhibitors of MET, and antibodies binding MET or its ligand HGF, Id. at 921-922. G. Smollen et al., “Amplification of MET may identify a subset of cancers with extreme sensitivity to the selective tyrosine kinase inhibitor PHA-665752”. Proc. Nat. Acad. Sci. (USA), 103(7): 2316-2321 (Feb. 14, 2006), which is incorporated herein by reference, report on the small molecule MET inhibitor PHA-665752. Birchmeier et al. and Smollen et al. do not disclose nor suggest any single nucleotide polymorphisms in the MET promoter as impacting effectiveness of MET receptor antagonist therapy.

A single nucleotide polymorphism (SNP) in the MET promotor has recently been identified as a risk factor for the development of autism, D. Campbell et al., “A genetic variant that disrupts MET transcription is associated with autism”, Proc. Nat. Acad. Sci. (USA) Early Edition, published Oct. 19, 2006, DOI: 10/1073/pnas.0605296103, pages 1-6 (hereafter cited as “Campbell”), which is incorporated herein by reference. Campbell et al. disclose that a certain SNP (the “rs1858830 G/C variant”) in the MET promotor resulted in a two fold decrease in MET transcription in two mouse neuronal cell lines and in a human embryonic kidney cell line. Campbell et al. do not disclose nor suggest assessment of this SNP for its impact on response to therapy using MET inhibitors.

Targeted cancer therapy is more often thought of as the use of diagnostic assays to identify patients that are more likely to respond to the therapy. However, because cancer patients often exhibit lower response to each subsequent therapy regime, it is essential to use a therapy with a better likelihood of effectiveness and to not use a therapy with a lower likelihood of response. Assays related to potential therapeutic use of MET receptor antagonists that would identify patients likely to exhibit lower response to MET receptor inhibition therapy are therefore needed.

### SUMMARY OF THE INVENTION

The invention provides assays for prediction of patients likely to exhibit lower response to MET inhibitor therapy. The inventive assays comprise assessment in a patient tissue sample of the presence of a single nucleotide polymorphism, the “rs185830-C allele”, in the promotor of the MET gene, located within human chromosome 7 between nucleotides 41495741 and 41496392. The SNP comprises substitution of a cytosine for a guanine in the sequence . . . GCG CTG GGC TCA GCC C GGC CGC AGG TGA CC . . . (SEQ. ID. NO. 1), where the mutation cytosine appears in bold. Presence of this promotor SNP can down regulate transcription of the MET gene, and if present, will likely lead to lower response to MET inhibitor therapy. The inventive methods preferably comprise assessment of the MET promotor SNP in solid tissue or blood samples by nucleic acid based assays.

In a preferred embodiment, the invention comprises a method for classifying a patient as likely to exhibit lower response to anti-MET-receptor therapy comprising: (a) providing a tissue sample from a patient; (b) determining MET promoter allele presence or absence in the patient tissue sample, wherein the MET promoter allele comprises a rs185830-C single nucleotide polymorphism; and (c) classifying the patient as likely to exhibit lower response to anti-MET receptor therapy where the patient sample is determined to comprise the rs185830-C polymorphism.

In another preferred embodiment, the invention comprises a nucleic acid based assay for the presence of the rs185830-C allele comprising: (a) providing a blood sample from a cancer patient; (b) extracting chromosomal DNA from the blood sample; (c) amplifying the chromosomal DNA by polymerase chain reaction using nucleic acid primers of sequence GATTTCCCTCTGGGTGGTG (SEQ. ID. NO. 2), as the forward primer, and CAAGCCCCATTCTAGTTTCG (SEQ. ID. NO. 3), as the reverse primer, to produce an amplified DNA sample; and (d) determining presence or absence of a rs185830-C allele in the amplified DNA sample. More preferably, the presence of absence of the allele is determined by a real-time PCR method, such as a Taqman assay. In this embodiment, it is preferred to use a pair of detector probes, which comprise the “G allele” detector probe CGCTGGGCTCAGCCGGG (SEQ. ID. NO. 4) and the “C allele” detector probe CTGGGCTCAGCCCGGCC (SEQ. ID. NO. 5), where the G allele probe is labeled on its 5′ end with fluorescein and on its 3′ end with a fluorescent quencher label, and the C allele probe is labeled on its 5′ end with the VIC® (a registered trademark of Applied Biosystems, Foster City, Calif.) fluorescent label and on its 3′ end with the fluorescent quencher label.

The invention has significant capability to provide improved selection of patients for anti-MET receptor therapy by identifying patients likely to exhibit lower response rates. The assessment of the presence of the MET promoter SNP implements personalized medicine, the classification of an individual patient based on the patient's likelihood of response to the therapy. The inventive assays have particular utility with any MET receptor antagonist therapy for treatment of cancer.

### DETAILED DESCRIPTION OF THE INVENTION

I. General

As used herein, MET (official symbol MET, also known as c-Met) means the human met proto-oncogene gene, which maps to 7q31; and HGF (official symbol HGF) means the human hepatocyte growth factor gene, which maps to 7q21.1. Chromosomal loci and chromosome 7 nucleotide numbers cited herein are based on Build 35 of the Human Genome Map, as accessed through the University of California Santa Cruz Genome Browser. As used herein, reference to a chromosome locus or band, such as 7q21, refers to all of the loci or sub bands, for example, such as 7q21.1, within the band.

The invention is based on the recognition by Applicant that a SNP identified in the MET receptor promoter, which leads to decreased transcription of the MET receptor, can lower the likelihood of response to MET receptor inhibitor therapy. As used herein, a “MET receptor antagonist” or “MET receptor inhibitor” refers to a therapeutic compound of any type including small molecule-, antibody-, antisense-, small interfering RNA- or microRNA-based compounds, that binds to the MET receptor or to the MET receptor ligand HGF and antagonizes the activity of signaling through the MET receptor. The inventive methods are useful with any known or hereafter developed MET receptor antagonist, and for example, are useful with cancer therapy comprising geldanmycin or the small molecule inhibitors SU11274 and PHA-665752.

MET receptor overexpression has been disclosed in multiple cancers, including bladder, breast, cervical, colorectal, esophageal, gastric, head and neck, kidney, liver, lung, nasopharyngeal, ovarian, pancreas, gall bladder, prostate and thyroid carcinomas, muscoskeletal sarcomas including osteosarcoma, synovial sarcoma, and rhabdomyosarcoma, soft tissue sarcomas including fibrososarcoma, leiomyosarcoma and Kaposi's sarcoma, hematopoetic malignancies including multiple myeloma, lymphomas, and adult T-cell leukemia, glioblastomas, astroycytomas, melanomas, mesotheliomas and Wilm's tumors. The invention has potential use with MET receptor inhibitor therapy for any of these cancers. In particular, the inventive assays are useful with MET receptor inhibitor therapy for cancers having chromosomal amplification at the MET receptor locus at 7q31, such as gastric carcinoma.

The invention comprises diagnostic assays performed on a patient tissue sample of any type or on a derivate thereof, including peripheral blood, tumor or suspected tumor tissues (including fresh frozen and fixed or paraffin embedded tissue), cell isolates such as circulating epithelial cells separated or identified in a blood sample, lymph node tissue, bone marrow and fine needle aspirates. A preferred tissue sample for use herein is a peripheral blood sample, because the SNP is more likely to be present as a germline mutation, as opposed to a somatic mutation that would require testing of tumor tissue.

II. MET Promoter Single Nucleotide Polymorphism

The invention comprises detection of the presence or absence of the “rs185830-C allele”, in the promotor of the MET gene, located within human chromosome 7 between nucleotides 41495741 and 41496392. In pertinent part, the MET promoter contains two allelic variations at one part of the promoter, the rs185830-C allele, which contains a cytosine in the pertinent sequence, and “the rs185830-G allele”, which contains a guanidine in place of cytosine. The pertinent sequence of the promoter and the two alleles are disclosed in Campbell. Campbell also disclose this mutation is located 20 base pairs 5′ to the MET transcription start site. The rs185830-C allele comprises in pertinent part the sequence GCG CTG GGC TCA GCC C GGC CGC AGG TGA CC . . . (SEQ. ID. NO. 1), with the SNP cytosine shown in bold in the sequence.

As disclosed in Campbell, the rs185830-C allele, if present, decreases transcription of MET by two fold in the mouse and human embryonic cell lines studied. The presence of the rs185830-C allele can therefore affect the effectiveness of therapy targeted at binding to the MET protein, because of the lowered transcription of MET. Applicant anticipates that the presence of the rs185830-C allele SNP would likely result in lowered response to MET receptor inhibition therapy. Hence, determination of the presence or absence of the rs185830-C allele in a cancer patient is helpful clinical information to be used in deciding whether to initiate MET receptor inhibitor therapy.

III. Assays

The inventive assays comprise assays believed to be predictive of lower response to MET receptor inhibitors, and preferably comprise nucleic acid based assay methods. Any suitable type of nucleic acid assays can be used. Nucleic acid assay methods useful in the invention are also well known in the art and comprise (i) PCR or other amplification assays to detect chromosomal DNA sequences; (ii) microarray hybridization assays to detect chromosomal DNA sequences, or (iii) nucleic acid sequencing methods. Assays using synthetic analogs of nucleic acids, such as peptide nucleic acids, in any of these formats can also be used.

Assays for detection of particular single nucleotide polymorphisms are known as “SNP genotyping assays”. PCR based reagents for SNP genotyping assays are commercially available from Applied Biosystems Incorporated (Foster City, Calif.) as products for SNP Genotyping Assays-On-Demand, for use with the ABI Prism 7900HT and SDS software, available from Applied Biosystems. Preferred assays comprise Taqman® (a trademark of Applied Biosystems) or real-time PCR assays, in which the amplification of the target DNA is monitoring during the amplification process. These assays are well known in the art. The detection probes used in real-time PCR or other amplification assays are preferably fluorescent.

These preferred assays use a pair of primers, the forward primer and the reverse primer, of any suitable sequence, for amplification by polymerase chain reaction of the promoter region of the MET receptor. The MET promoter is described in Campbell et al. and its sequence can be preferably be amplified for use in the invention by using a pair of primers to generate a 652 base pair long amplicon containing the target SNP locus. Id. The sequence of each of these primers is GATTTCCCTCTGGGTGGTG (SEQ. ID. NO. 2), the forward primer, and CAAGCCCCATTCTAGTTTCG (SEQ. ID. NO. 3), the reverse primer. These primers are preferably used with 5% DMSO and an annealing temperature of 61 degrees C., as disclosed by Campbell.

As disclosed by Campbell, the 200-bp fragment of genomic DNA immediately surrounding the rs1858830 locus is very GC-rich: ˜85% of the nucleotides are either G or C, and does not amplify well. Design of primers and detector probes for use in the invention should take this into consideration.

It is preferred to use a pair of Taqman® detector probes, which comprise the “G allele” detector probe CGCTGGGCTCAGCCGGG (SEQ. ID. NO. 4) and the “C allele” detector probe CTGGGCTCAGCCCGGCC (SEQ. ID. NO. 5), where the G allele probe is labeled on its 5′ end with fluorescein and on its 3′ end with a BHQ (Black Hole Quencher) dye (available from Biosearch Technologies, Novato, Calif.), and the C allele probe is labeled on its 5′ end with the VIC® (a registered trademark of Applied Biosystems, Foster City, Calif.) fluorescent label and on its 3′ end a BHQ label.

A preferred real-time PCR reagent and target reaction mixture comprises:

PCR buffer (50 mM KCl, 15 mM Tris-HCl, pH=8.0)

1-5 mM MgCl₂

1-5 ng genomic DNA

0.75-1.5 units DNA polymerase (AmpliTaq Gold (Applied Biosystems)

100 μM dNTP mix

100-200 nM forward primer

100-200 nM reverse primer

100-200 nM G-allele probe

100-200 nM C-allele probe,

with a 25-50 μl total reaction mixture volume.

Useful real-time PCR assay conditions comprise performing the first amplification cycle at 95° C. for 5-10 minutes (polymerase activation), then 95° C. for 15 seconds (template melt), then 70-72° C. for 30-60 seconds (anneal/extension), with these alternating cycles repeated for 30 to 40 cycles, while monitoring the fluorescein and VIC® fluorescence.

### IV. Sample Processing

The preferred tissue samples for use herein are peripheral blood samples. Tumor or suspected tumor tissue can also be used. The tissue sample can be processed by any suitable method, including conventional methods known in the art for extraction and purification of chromosomal DNA for use in nucleic acid based assays. Multiple chromosomal DNA extraction kits are available commercially, including the QIAamp blood kit (QIAGEN, Inc., Valencia, Calif.) and the Puregene DNA isolation kit (Gentra Systems, Inc., Minneapolis, Minn.).

### V. Instrumentation

Any suitable instrumentation or automation can be used in the performance of the inventive assays. Preferably, automation for performance of DNA extraction and real-time PCR analysis of the tissue sample are used. Real-time PCR detection instruments are available from Applied Biosystems. More preferably, the m2000sp automated DNA extraction instrument and the m2000rt automated real-time PCR instrument, available from Abbott Molecular (Des Plaines, Ill.), are use to carry out the inventive assays.

| | |
|---|---|
| <200> SEQUENCE CHARACTERISTICS: | |
| <210> SEQ ID NO: 1 | |
| <211> LENGTH: 30 | |
| <212> TYPE: DNA | |
| <213> ORGANISM: Artificial | |
| <220> FEATURE: | |
| <221> NAME/KEY: Misc_feature | |
| <222> LOCATION: 1-30 | |
| <223> OTHER INFORMATION: sequence is synthesized | |
| <400> SEQUENCE: 1 | |
| GCGCTGGGCT CAGCCCGGCC GCAGGTGACC | 50 |
| <200> SEQUENCE CHARACTERISTICS: | |
| <210> SEQ ID NO: 2 | |
| <211> LENGTH: 19 | |
| <212> TYPE: DNA | |
| <213> ORGANISM: Artificial | |
| <220> FEATURE: | |
| <221> NAME/KEY: Misc_feature | |
| <222> LOCATION: 1-19 | |
| <223> OTHER INFORMATION: sequence is synthesized | |
| <400> SEQUENCE: 2 | |
| GATTTCCCTC TGGGTGGTG | |
| 200> SEQUENCE CHARACTERISTICS: | |
| <210> SEQ ID NO: 3 | |
| <211> LENGTH: 20 | |
| <212> TYPE: DNA | |
| <213> ORGANISM: Artificial | |
| <220> FEATURE: | |
| <221> NAME/KEY: Misc_feature | |
| <222> LOCATION: 1-20 | |
| <223> OTHER INFORMATION: sequence is synthesized | |
| <400> SEQUENCE: 3 | |
| CAAGCCCCAT TCTAGTTTCG | |
| <200> SEQUENCE CHARACTERISTICS: | |
| <210> SEQ ID NO: 4 | |
| <211> LENGTH: 17 | |
| <212> TYPE: DNA | |
| <213> ORGANISM: Artificial | |
| <220> FEATURE: | |
| <221> NAME/KEY: Misc_feature | |
| <222> LOCATION: 1-17 | |
| <223> OTHER INFORMATION: sequence is synthesized | |
| <400> SEQUENCE: 4 | |
| CGCTGGGCTC AGCCGGG | |
| <200> SEQUENCE CHARACTERISTICS: | |
| <210> SEQ ID NO: 5 | |
| <211> LENGTH: 17 | |
| <212> TYPE: DNA | |
| <213> ORGANISM: Artificial | |
| <220> FEATURE: | |
| <221> NAME/KEY: Misc_feature | |
| <222> LOCATION: 1-17 | |
| <223> OTHER INFORMATION: sequence is synthesized | |
| <400> SEQUENCE: 5 | |
| CTGGGCTCAG CCCGGCC | |

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

What is claimed is:

1. A method for classifying a patient as likely to exhibit lower response to anti-MET-receptor therapy comprising: (a) providing a tissue sample from a patient; (b) determining MET promoter allele presence or absence in the patient tissue sample, wherein the MET promoter allele comprises a rs185830-C single nucleotide polymorphism; and (c) classifying the patient as likely to exhibit lower response to anti-MET receptor therapy where the patient's sample is determined to comprise the rs185830-C polymorphism.

2. The method of claim 1 wherein the tissue sample is a peripheral blood sample from a patient with a cancer selected from the group consisting of bladder, breast, cervical, colorectal, esophageal, gastric, head and neck, kidney, liver, lung, nasopharyngeal, ovarian, pancreas, gall bladder, prostate and thyroid carcinoma; muscoskeletal sarcoma including osteosarcoma, synovial sarcoma, and rhabdomyosarcoma; soft tissue sarcoma including fibrososarcoma, leiomyosarcoma and Kaposi's sarcoma; hematopoetic malignancy including multiple myeloma, lymphoma, and adult T-cell leukemia; glioblastoma; astroycytoma; melanoma; mesothelioma; and Wilm's tumor.

3. The method of claim 1 wherein the tissue sample is a peripheral blood sample from a patient with a cancer.

4. The method of claim 3 wherein the cancer is an epithelial cell based cancer.

5. The method of claim 1 wherein the MET promoter allele presence or absence is determined by a nucleic acid based assay using a pair of amplification primers comprising:

| | |
|---|---|
| GATTTCCCTCTGGGTGGTG, | (SEQ. ID. NO. 2) |
| and | |
| CAAGCCCCATTCTAGTTTCG. | (SEQ. ID. NO. 3) |

6. The method of claim 1 wherein the MET promoter allele is located on human chromosome 7, between nucleotides 41495741 and 41496392.

7. The method of claim 1 wherein the anti-MET receptor therapy comprises a small molecule inhibitor of MET, a small molecule inhibitor of HCF, an antibody inhbitor of MET, an antibody inhibitor of HCF, an siRNA inhibitor of MET, or an siRNA inhibitor of HCF.

8. The method of claim 5 wherein the MET promoter allele presence or absence is determined by real-time polymerase chain reaction using a pair of detector probes comprising the following sequences: CGCTGGGCTCAGCCGGG (SEQ. ID. NO. 4) and CTGGGCTCAGCCCGGCC (SEQ. ID. NO. 5).

9. A method for classifying a patient as likely to exhibit lower response to anti-MET-receptor therapy based on presence of a rs185830-C allele comprising: (a) providing a blood sample from a cancer patient; (b) extracting chromosomal DNA from the blood sample; (c) amplifying the chromosomal DNA by polymerase chain reaction using nucleic acid primers of sequence GATTTCCCTCTGGGTGGTG (SEQ. ID. NO. 2), and CAAGCCCCATTCTAGTTTCG (SEQ. ID. NO. 3) to produce an amplified DNA sample; and (d) determining presence or absence of a rs185830-C allele in the amplified DNA sample.

10. The method of claim 9, wherein the presence or absence of the rs185830 allele is determined by real-time PCR.

11. The method of claim 10 wherein a pair of detector probes comprising the following sequences: CGCTGGGCTCAGCCGGG (SEQ. ID. NO. 4) and CTGGGCTCAGCCCGGCC (SEQ. ID. NO. 5), are used.


SEQUENCE LISTING (5 sequences)

SEQ ID NO: 1 - DNA, 30 bp, Artificial Sequence
  Description of Artificial Sequence Synthetic oligonucleotide
gcgctgggctcagcccggccgcaggtgacc

SEQ ID NO: 2 - DNA, 19 bp, Artificial Sequence
  Description of Artificial Sequence Synthetic primer
gatttccctctgggtggtg

SEQ ID NO: 3 - DNA, 20 bp, Artificial Sequence
  Description of Artificial Sequence Synthetic primer
caagccccattctagtttcg

SEQ ID NO: 4 - DNA, 17 bp, Artificial Sequence
  Description of Artificial Sequence Synthetic probe
cgctgggctcagccggg

SEQ ID NO: 5 - DNA, 17 bp, Artificial Sequence
  Description of Artificial Sequence Synthetic probe
ctgggctcagcccggcc